(19) United States
(12) Patent Application Publication
(10) Pub. No.: US 2022/0167904 A1
(43) Pub. Date: Jun. 2, 2022

(54) ATRIAL FIBRILLATION ANALYTICAL APPARATUS, ATRIAL FIBRILLATION ANALYTICAL METHOD, AND STORAGE MEDIUM

(71) Applicants: NATIONAL UNIVERSITY CORPORATION TOKYO MEDICAL AND DENTAL UNIVERSITY, Tokyo (JP); KONICA MINOLTA, INC., Tokyo (JP)

(72) Inventors: Tetsuo Sasano, Bunkyo-ku, Tokyo (JP); Kanae Sasaki, Bunkyo-ku, Tokyo (JP)

(73) Assignees: NATIONAL UNIVERSITY CORPORATION TOKYO MEDICAL AND DENTAL UNIVERSITY, Tokyo (JP); KONICA MINOLTA, INC., Tokyo (JP)

(21) Appl. No.: 17/437,254

(22) PCT Filed: Dec. 23, 2019
(86) PCT No.: PCT/JP2019/050291

§ 371 Date: Sep. 8, 2021

(30) Foreign Application Priority Data
Mar. 8, 2019 (JP) 2019-042717

(51) Int. Cl. A61B 5/361; A61B 5/353; A61B 5/339

(52) U.S. Cl. CPC A61B 5/361; A61B 5/339; A61B 5/353

(57) ABSTRACT

An atrial fibrillation analysis device includes: a hardware processor that: acquires P-wave data from only one of either: a first electrocardiogram in a lead of one direction on a plane including a body-axis direction and a left-right direction with respect to a subject, or a second electrocardiogram in leads of two directions orthogonal to each other on the plane; extracts P-wave fragments from the acquired P-wave data; and analyzes a possibility of development of atrial fibrillation based on at least one of a number of the P-wave fragments and a duration of the P-wave fragments.

### BACKGROUND

### Technical Field

The present invention relates to an atrial fibrillation analysis device, an atrial fibrillation analysis method, and a storage medium storing instructions.

### Description of Related Art

Atrial fibrillation (AF) is the most common persistent arrhythmia disease. As atrial fibrillation causes cerebral infarction and heart failure, early diagnosis thereof is important. In addition, atrial fibrillation is arrhythmia that initially occurs as paroxysms and gradually becomes chronic. Conventionally, atrial fibrillation could be diagnosed by observing an electrocardiogram at the time of stroke, but could not be diagnosed by observing an electrocardiogram while no symptoms are presented. Therefore, it has been desired that the possibility of development of atrial fibrillation can be analyzed using electrocardiography, which is a non-invasive examination, while no symptoms are presented.

Under such circumstances, Non-Patent Literature 1 discloses a technique to count fragments of P-waves on an electrocardiogram after bandpass filtering as a new analysis method for evaluating conduction in the atrium.

### Non-Patent Literature

Non-Patent Literature 1: Murthy S, Rizzi P, Mewton N, Strauss D G, Liu C Y, Volpe G J, Marchlinski F E, Spooner P, Berger R D, Kellman P, Lima J A C, Tereshchenko L G. “Number of P-Wave Fragmentations on P-SAECG Correlates with Infiltrated Atrial Fat”, Ann Noninvasive Electrocardiol 2014; 19: 114-121.

However, in Non-Patent Literature 1, only the correlation between the P-wave fragments and the interatrial septal fat is observed, and the correlation with the atrial fibrillation development prediction is not evaluated. Moreover, in Non-Patent Literature 1, an XYZ-lead electrocardiogram of a special lead system (Frank Lead) is used, and a 12-lead electrocardiogram, which is commonly used in clinical practice, is not used. Therefore, it requires an expensive device and an inspector with high skills, and there is a large obstacle for bringing it into widespread use. Regarding analysis using an electrocardiogram of a special lead system, it is difficult to improve the accuracy of analysis of the possibility of development of atrial fibrillation because an amount of data of electrocardiograms measured in the past is small.

### SUMMARY

One or more embodiments of the present invention enable accurate determination of the possibility of development of the atrial fibrillation with a non-invasive, inexpensive, easy, and short-time examination, even while no symptoms are presented.

An atrial fibrillation analysis device of one or more embodiments includes:

a P-wave data acquirer (i.e., a hardware processor) that acquires P-wave data from only one of either: a first electrocardiogram in a lead of one direction on a plane including a body-axis direction and a left-right direction with respect to a subject, or a second electrocardiogram in leads of two directions orthogonal to each other on the plane;

a fragment extractor (i.e., the hardware processor) that extracts P-wave fragments from the P-wave data acquired by the P-wave data acquirer; and

an analyzer (i.e., the hardware processor) that analyzes a possibility of development of atrial fibrillation based on at least one of a number of the P-wave fragments and a duration of the P-wave fragments.

According to one or more embodiments, the atrial fibrillation analysis device further includes: an ECG measurer that measures the first or second electrocardiogram.

According to one or more embodiments, the P-wave data acquirer acquires a plurality of pieces of P-wave data from first or second the electrocardiogram, and the fragment extractor averages the plurality of pieces of P-wave data to calculate averaged P-wave data, extracts an extreme value from the averaged P-wave data, and in response to a potential difference between the extreme value and an adjacent extreme value exceeding a predetermined value, extracts a line connecting the extreme value and the adjacent extreme value as a P-wave fragment.

According to one or more embodiments, in response to acquisition of the P-wave data from the second electrocardiogram in the two leads orthogonal on the plane by the P-wave data acquirer, the fragment extractor calculates the averaged P-wave data by averaging the plurality of pieces of P-wave data for each of the leads to calculate a root mean square.

According to one or more embodiments, the fragment extractor filters out a predetermined range of frequency from the averaged P-wave data and extracts P-wave fragments from the filtered averaged P-wave data.

An atrial fibrillation analysis method of one or more embodiments includes:

acquiring P-wave data from only one of either: an electrocardiogram in a lead of one direction on a plane including a body-axis direction and a left-right direction with respect to a subject, or an electrocardiogram in leads of two direction orthogonal to each other on the plane;

extracting P-wave fragments from the P-wave data acquired by the P-wave data acquirer; and

analyzing a possibility of development of atrial fibrillation based on at least one of a number of the P-wave fragments and a duration of the P-wave fragments.

A non-transitory computer-readable storage medium of one or more embodiments stores instructions that cause a computer to function as:

a P-wave data acquirer that acquires P-wave data from only one of either: an electrocardiogram in a lead of one direction on a plane including a body-axis direction and a left-right direction with respect to a subject, or an electrocardiogram in leads of two direction orthogonal to each other on the plane;

a fragment extractor that extracts P-wave fragments from the P-wave data acquired by the P-wave data acquirer; and

an analyzer that analyzes a possibility of development of atrial fibrillation based on at least one of a number of the P-wave fragments and a duration of the P-wave fragments.

According to one or more embodiments, it is possible to accurately determine the possibility of development of atrial fibrillation with a non-invasive, inexpensive, easy, and short-time examination, even while no symptoms are presented. As a result, early diagnosis of atrial fibrillation is possible.

### BRIEF DESCRIPTION OF DRAWINGS

FIG. 1 is a block diagram showing a functional configuration of an atrial fibrillation analysis device in one or more embodiments of the present invention.

FIG. 2 is a flowchart showing a flow of an atrial fibrillation analysis process A executed by a controller in FIG. 1 in the first embodiment.

FIG. 3 is an explanatory diagram of a waveform of an electrocardiogram.

FIG. 4A shows leads X and Y of an XYZ-lead ECG machine.

FIG. 4B shows limb leads of a 12-lead ECG machine.

FIG. 5 shows a flow of calculating the number and duration of P-wave fragments from the average P-wave data.

FIG. 6A shows an example of the number and duration of P-wave fragments of a healthy person.

FIG. 6B is an example of the number and duration of P-wave fragments of a patient with paroxysmal atrial fibrillation.

FIG. 7A shows a result of comparison of the numbers of P-wave fragments in leads XY and the numbers of P-wave fragments in leads XYZ.

FIG. 7B shows a result of comparison of the durations of P-wave fragments in leads XY and the durations of P-wave fragments in leads XYZ.

FIG. 8A is a scatter plot showing correlations between the numbers of P-wave fragments in leads XY and the numbers of P-wave fragments in leads I and aVF of a 12-lead ECG machine.

FIG. 8B is a scatter plot showing correlations between the durations of P-wave fragments in leads XY and the durations of P-wave fragments in leads I and aVF of a 12-lead ECG machine.

FIG. 9A is a scatter plot showing correlations between the numbers of P-wave fragments in leads I and aVF of a 12-lead ECG machine and the numbers of P-wave fragments in leads II and aVL.

FIG. 9B is a scatter plot showing correlations between the durations of P-wave fragments in leads I and aVF of a 12-lead ECG machine and the durations of P-wave fragments in leads II and aVL.

FIG. 10A is a scatter plot showing correlations between the numbers of P-wave fragments in leads I and aVF of a 12-lead ECG machine and the numbers of P-wave fragments in leads III and aVR.

FIG. 10B is a scatter plot showing correlations between the durations of P-wave fragments in leads I and aVF of a 12-lead ECG machine and the durations of P-wave fragments in leads III and aVR.

FIG. 11 is a flowchart showing a flow of the atrial fibrillation analysis process B executed by a controller in FIG. 1 in the second embodiment.

### DETAILED DESCRIPTION OF EMBODIMENTS

Hereinafter, embodiments of the present invention are described with reference to the drawings. However, the scope of the present invention is not limited to the embodiments or illustrated examples.

### First Embodiment

An example of analysis of the possibility of development of atrial fibrillation is described in the first embodiment, where an electrocardiogram of two leads orthogonal on a plane (coronal plane) including a body-axis direction (cephalocaudal direction) and a left-right direction with respect to the subject is only used, and an electrocardiogram in a lead in the anteroposterior direction with respect to the body is not used.

### [Configuration of Atrial Fibrillation Analysis Device 1]

First, the configuration of the atrial fibrillation analysis device 1 in the first embodiment of the present invention is described.

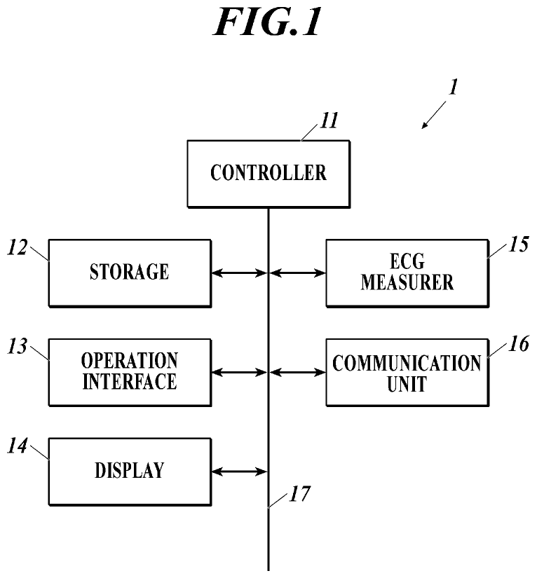

FIG. 1 is a block diagram showing a functional configuration of the atrial fibrillation analysis device 1. As shown in FIG. 1, the atrial fibrillation analysis device 1 includes a controller (i.e., a hardware processor) 11, a storage 12, an operation interface 13, a display 14, an ECG measurer 15, and a communication unit 16, and the components are connected with each other by a bus 17. This embodiment shows an atrial fibrillation analysis device with an ECG measurer, but an atrial fibrillation analysis device without an ECG measurer may also be used. In an atrial fibrillation analysis device without an ECG measurer, data of electrocardiograms is stored in the storage via the communication unit or the like, and an atrial fibrillation analysis process may be performed based on the data of electrocardiograms stored in the storage.

The controller 11 includes a central processing unit (CPU), and a random access memory (RAM). In response to the operation of the operation interface 13, the CPU of the controller 11 reads out system instructions and various types of processing instructions stored in the storage 12, loads them to the RAM, and centrally controls the operations of the components in the atrial fibrillation analysis device 1 in accordance with the loaded instructions. For example, the controller 11 executes the atrial fibrillation analysis device described later in response to the operation of the operation interface 13, and functions as a P-wave data acquirer, a fragment extractor, and an analyzer.

The storage 12 includes a non-volatile semiconductor memory, a hard disk, and the like. The storage 12 stores the system instructions and the various types of instructions to be executed by the controller 11, and data such as parameters necessary for the processing by the instructions. For example, the storage 12 stores an instruction for executing the atrial fibrillation analysis process described later. The storage 12 stores data of electrocardiograms. The various types of instructions are stored in a form of readable program code, and the controller 11 sequentially executes the operation according to the program code.

The operation interface 13 includes various function keys and a pointing device such as a mouse. The operation interface 13 outputs, to the controller 11, an instruction signal which is input by a key operation and a mouse operation performed by the user. The operation interface 13 may include a touch panel on the display screen of the display 14. In this case, the operation interface 13 outputs an instruction signal input via the touch panel to the controller 11.

The display 14 is constituted of a monitor such as a liquid crystal display (LCD), and a cathode ray tube (CRT), and displays input commands, data, and the like from the operation interface 13, according to the commands of the display signals input from the controller 11.

The ECG measurer 15 measures electrical changes of the myocardium via electrodes arranged on the body surface of the subject and records them as an electrocardiogram. A 12-lead ECG machine which is widely used may be used as the ECG measurer 15, but an XYZ-lead ECG machine may also be used.

The communication unit 16 includes a LAN adapter, a modem, and a terminal adapter (TA), and controls data transmission and reception to and from an external device(s) connected to the communication network.

### [Actions of Atrial Fibrillation Analysis Device 1]

Next, the actions of the atrial fibrillation analysis device 1 in this embodiment is described.

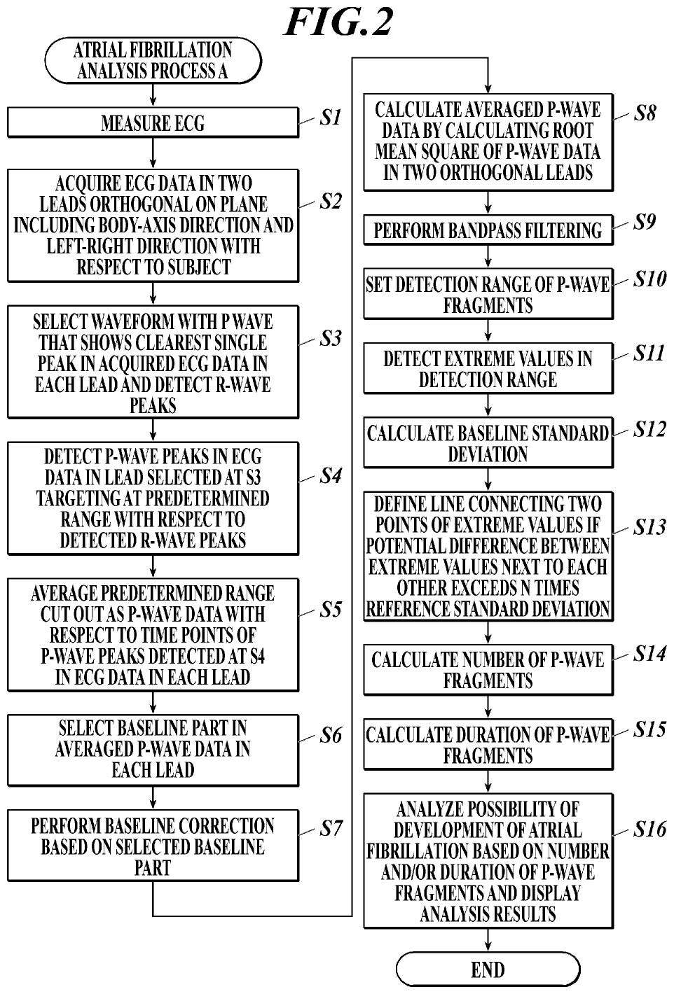

FIG. 2 is a flowchart showing a flow of the atrial fibrillation analysis process (referred to as the atrial fibrillation analysis process A) executed by the controller 11 of the atrial fibrillation analysis device 1. The atrial fibrillation analysis process A is executed by the CPU of the controller 11 in cooperation with the instruction stored in the storage 12 according to the operation of the operation interface 13.

First, the controller 11 causes the ECG measurer 15 to measure an electrocardiogram of the subject to record digital data of electrocardiograms (electrocardiogram data) in a sinus rhythm (while no symptoms are presented) (Step S1).

In order to maintain the accuracy of analysis, the measurement time of an electrocardiogram is preferably 10 seconds or more and 1 hour or less. More preferably, the measurement time is 10 seconds and more and 30 minutes or less, and most preferably, 10 seconds or more and 3 minutes or less. The analysis method of the atrial fibrillation analysis device 1 may be used because it is not necessary to perform long-time measurement such as 24-hour measurement using Holter ECG and because it is possible to analyze the possibility and risk of development of atrial fibrillation by short-time measurement. In this embodiment, 100 heartbeats are measured in two minutes.

In the analysis of this embodiment, as a lead in the front-back direction with respect to the body is not used, measurement in a lead in the front-back direction may be omitted.

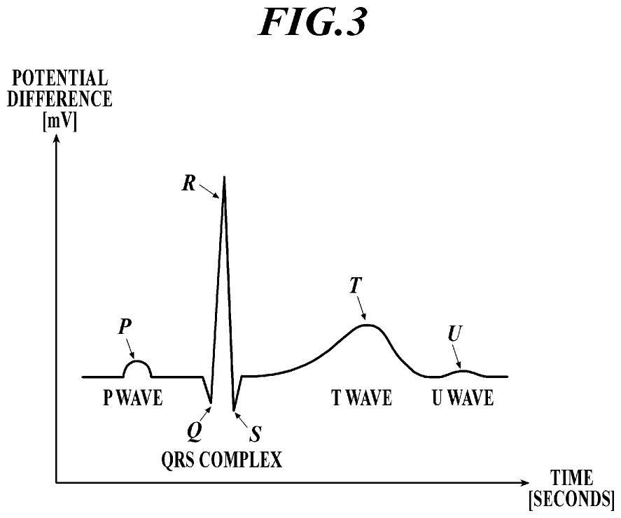

FIG. 3 shows an example of data of an electrocardiogram of one heartbeat. As shown in FIG. 3, the ECG data consists of P wave, Q wave, R wave, S wave, (QRS complex), T wave, and U wave. The horizontal axis indicates the time axis (mS) and the vertical axis indicates the potential difference (mV).

Next, the controller 11 acquires ECG data in two leads orthogonal on the plane including the body-axis direction and the left-right direction with respect to the body from the ECG data acquired by the ECG measurer 15 (Step S2).

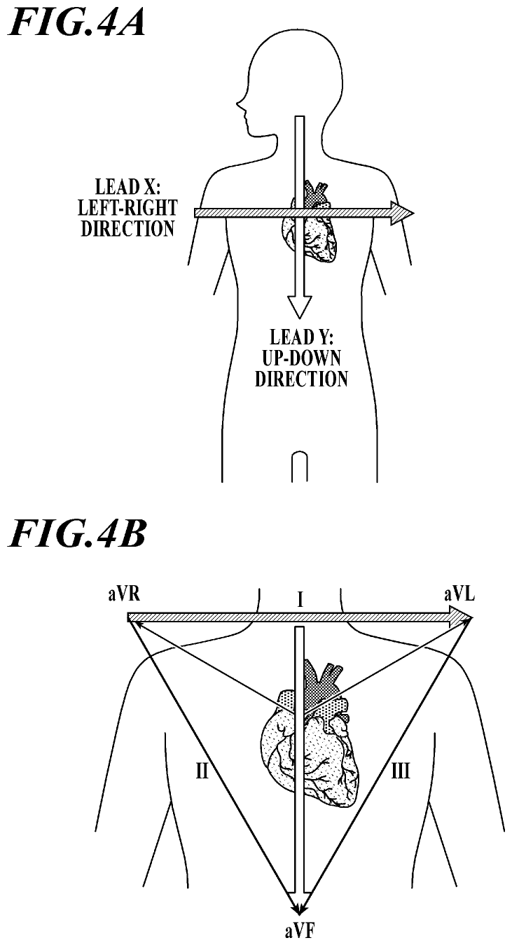

FIG. 4A shows leads X and Y of an XYZ-lead ECG machine. FIG. 4B shows limb leads of a 12-lead ECG machine. As shown in FIGS. 4A and 4B, two leads orthogonal on the plane including the body-axis direction (cephalocaudal direction, vertical direction) and the left-right direction with respect to the subject are leads X and Y of an XYZ-lead ECG machine (hereinafter, leads XY), and lead I and lead aVF, lead II and lead aVL, lead III and lead aVR of limb leads of the 12-lead ECG machine. In a case where the ECG measurer 15 is a 12-lead ECG machine, the controller 11 acquires the ECG data in the above-described pairs of leads among the ECG data of limb leads (in this embodiment, all ECG data of limb leads). In a case where the ECG measurer 15 is an XYZ-lead ECG machine, ECG data in the leads X and Y is acquired.

Next, the controller 11 selects ECG data of a waveform whose P wave shows the clearest single peak in the acquired ECG data of each lead and detects an R-wave peak in the selected ECG data (Step S3).

At Step S3, for example, the acquired ECG data in each lead is serially displayed on the display 14, and the ECG data including the waveform whose P wave shows the clearest single peak may be selected by a user operation. Alternatively, the controller 11 may automatically select the forms and heights of the waves included in the ECG data in each lead.

For example, in a case where ECG data of 100 heartbeats is recorded, 100 R-wave peaks are detected in the selected ECG data. The R-wave peaks detected here may be part of multiple heartbeats acquired in one-time ECG measurement, but are R-wave peaks of all the heartbeats in one or more embodiments. The P-wave peaks and the R-wave peaks included in the ECG data can be automatically detected by the controller 11 based on the waveform.

Next, the controller 11 detects P-wave peaks in the ECG data in leads selected at Step S3, targeting a predetermined range with each R-wave peak detected in the selected ECG data as a reference (Step S4).

The predetermined range targeted for detection of P-wave peaks is defined experimentally and empirically as a range where P-wave peaks exist, which is a range of −50 to −200 mS with respect to each R-wave peak, for example,

In the case where ECG data with 100 heartbeats is recorded, 100 P-wave peaks are detected from the ECG data in each lead, for example.

Next, the controller 11 acquires the predetermined range with time points of P-wave peaks detected at Step S4 as references in the ECG data in each lead as the P-wave data, and averages them (Step S5).

The P-wave data cut out here are data on at least part of multiple heartbeats acquired in one-time ECG measurement, or all the heartbeats in one or more embodiments. The number of P waves in the data is preferably 100 or more, more preferably 500 or more, and most preferably 1000 or more.

The predetermined range cut out as the P-wave data is defined experimentally and empirically as a range of P waves and baselines before and after the P waves, which is, for example, a range of −500 mS to +300 mS with respect to the P-wave peak including 0 mS with the absolute value of the minus limit being larger than the absolute value of the plus limit, a range of −300 mS to +150 mS with respect to each P-wave peak. The baseline is the part of the ECG data where the heart is not excited.

Next, the controller 11 selects the baseline part from the P-wave data of each lead averaged (Step S6).

For example, the controller 11 selects the predetermined range as the baseline with respect to each P-wave peak (for example, −200 to −100 mS). The range selected as the baseline is defined experimentally and empirically as a range where the baseline exists. The baseline part may be selected by the user.

Next, the controller 11 performs baseline correction of the P-wave data averaged based on the selected baseline part (Step S7).

For example, the average of the selected baseline part is calculated and subtracted from the values of the P-wave data to perform the baseline correction. As a result of this, the value of the baseline part can be almost 0.

Next, the controller 11 calculates the root mean square (RMS) of the P-wave data in two orthogonal leads to calculate the averaged P-wave data (Step S8).

For example, in the case where the ECG measurer 15 is a 12-lead ECG machine, the root mean squares of three pairs of the P-wave data of lead I and lead aVF, lead II and lead aVL, and lead III and lead aVR are calculated to obtain the three pairs of the averaged P-wave data. Alternatively, the root mean square of the P-wave data of lead I and lead aVF is calculated to obtain one pair of the averaged P-wave data only.

In the case where the ECG measurer 15 is an XYZ-lead ECG machine, the root mean square of the P-wave data of leads X and Y is calculated to obtain one pair of the averaged P-wave data.

Next, bandpass filtering is performed on the calculated averaged P-wave data (Step S9). The frequency range to pass is obtained experimentally and empirically, and is preferably 30 to 300 Hz and more preferably 40 to 150 Hz.

Next, the controller 11 sets the detection range of a P-wave fragment in the averaged P-wave data after the bandpass filtering (Step S10).

For example, the part right after the baseline part selected at Step S6 and right before the QRS complex in the averaged P-wave data after the bandpass filtering is set as the detection range of a P-wave fragment.

Next, the controller 11 detects the extreme values in the detection range of a P-wave fragment (Step S11).

Next, the controller 11 calculates the standard deviation (baseline standard deviation) of the values in the baseline part of the averaged P-wave data after the bandpass filtering (Step S12).

This baseline standard deviation indicates a noise level when the electrocardiogram is measured.

Here, in the case where the ECG measurer 15 is a 12-lead ECG machine, the standard average is calculated from the three sets of the averaged P-wave data, and the averaged P-wave data with the smallest standard deviation, namely with the least noise, is defined as the waveform for calculating the P-wave fragment. Alternatively, the standard deviation may be calculated from the averaged P-wave data in lead I and lead aVF only, not using the three sets of the averaged P-wave data.

Next, if the potential difference between the extreme values next to each other detected at Step S11 exceeds n times the baseline standard deviation (n is a positive number), the controller 11 defines the line connecting the two points as a P-wave fragment (Step S13).

n is a value calculated based on the experiment, and is preferably 2 or more and 10 or less, and more preferably 2 or more and 5 or less. n is 3, for example.

Next, the controller 11 calculates the number of P-wave fragments (Step S14).

Next, the controller 11 calculates the time (duration of the P-wave fragments) from the start point (the first start point in the single averaged P-wave data) to the end point (the last end point in the single averaged P-wave data) of the P-wave fragments (Step S15).

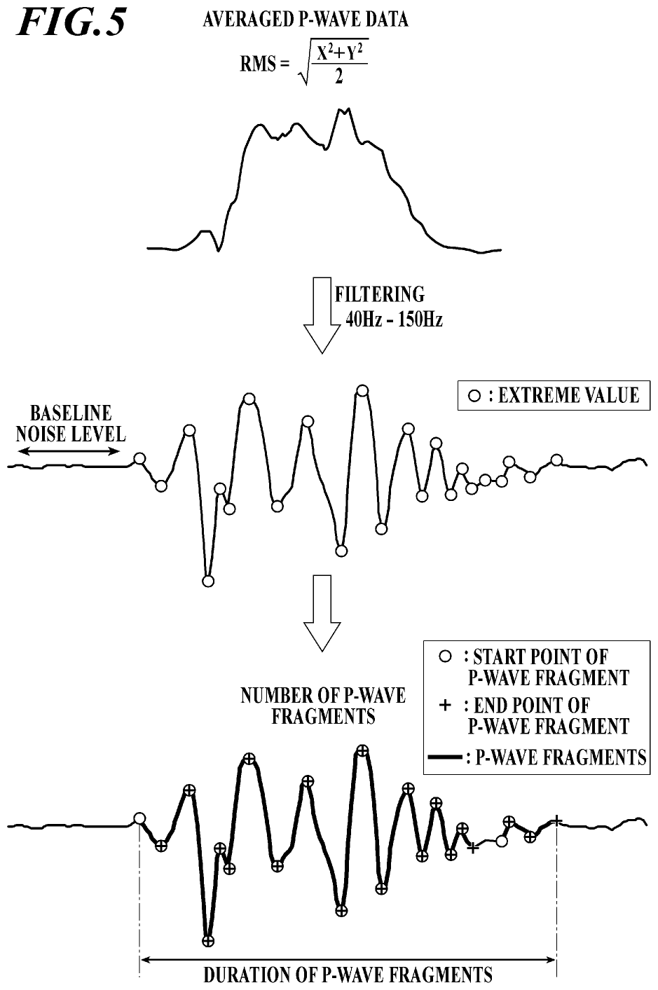

FIG. 5 shows a flow of calculating the number and the duration of P-wave fragments from the averaged P-wave data.

The controller 11 analyzes the possibility of development of atrial fibrillation based on the number and/or duration of P-wave fragments, displays the analysis results on the display 14 (Step S16), and ends the atrial fibrillation analysis process A.

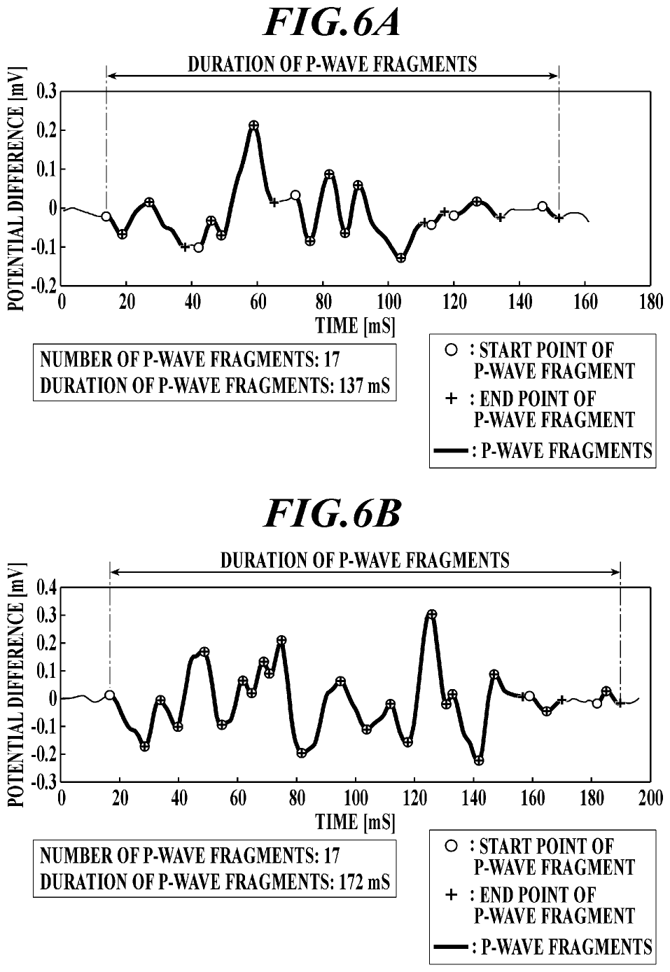

FIG. 6A shows an example of the number and duration of P-wave fragments of a healthy person, and FIG. 6B is an example of the number and duration of P-wave fragments of a patient with symptomatic atrial fibrillation. In this embodiment, n is 3. That is, in this embodiment, if a potential difference between the extreme values next to each other exceeds three times the baseline standard deviation, the line connecting the two points is defined as a P-wave fragment. As shown in FIGS. 6A and 6B, the number and duration of P-wave fragments of a patient with paroxysmal atrial fibrillation is larger than those of a healthy person. In this embodiment, the number of P-wave fragments of a healthy person is 17, and the number of P-wave fragments of a patient with paroxysmal atrial fibrillation is 25. The time (duration) of P-wave fragments of a healthy person is 137 ms, and the time (duration) of P-wave fragments of a patient with paroxysmal atrial fibrillation is 172 ms.

At Step S16, for example, the number or duration of P-wave fragments is shown as an index of the possibility of development of atrial fibrillation. Alternatively, thresholds of the number or duration of P-wave fragments may be set, and if the calculated number or duration of P-wave fragments is larger than a threshold, it is determined and shown that the possibility of development of atrial fibrillation is high. Alternatively, it may be determined and shown that the possibility of development of atrial fibrillation is low if the number or duration of P-wave fragments is Threshold 1 or less; that the possibility is medium if the number or duration is Threshold 1 to less than Threshold 2; and that the possibility is high if the number or duration is Threshold 2 or more (Threshold 1<Threshold 2). Alternatively, for example, a table in which combinations of the number and duration of P-wave fragments are associated with the indexes of the possibility of development of atrial fibrillation may be stored beforehand in the storage 12, and an index associated with a combination of the calculated number and duration of P-wave fragments may be read out and shown.

### [Evaluation]

The inventors of the present invention supposed, as a result of painstaking research, that a lead in the front-back direction with respect to the body is not necessary for analysis of the possibility of development of atrial fibrillation because the left atrium posterior wall is an important region in occurrence of atrial fibrillation. They evaluated whether it is possible to use, in determination of the possibility of development of paroxysmal atrial fibrillation, the calculated number and duration of P-wave fragments measured only in two leads orthogonal on the plane including the body-axis direction and the left-right direction without lead in the front-back direction with respect to the body while no symptoms are presented.

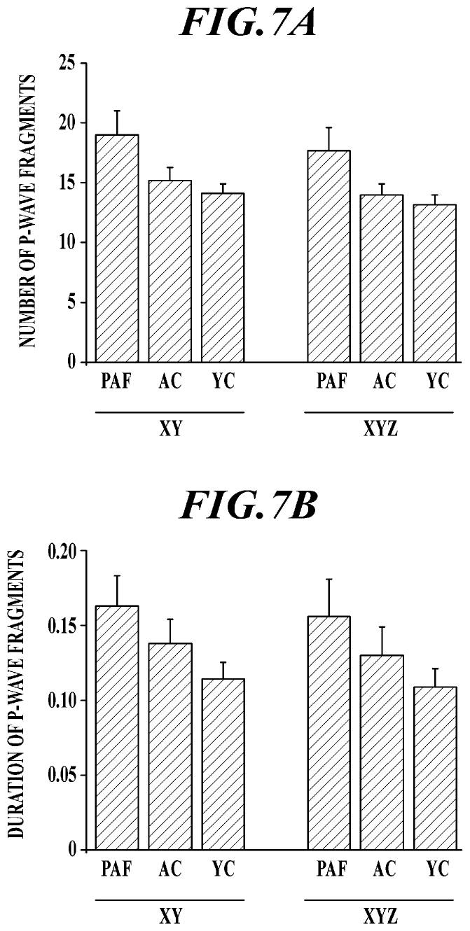

FIG. 7A shows results of comparison of the numbers of P-wave fragments (average) calculated by the above-described method from two-minute recording in leads XY and leads XYZ in PAF (paroxysmal atrial fibrillation group), AC (age-matched control group), and YC (young-age control group). FIG. 7B shows results of comparison of the durations of P-wave fragments (average) calculated by the above-described method from two-minute recording in leads XY and leads XYZ in PAF, AC, and YC. The threshold for defining P-wave fragments is three times the noise level at the baseline part.

As shown in FIGS. 7A and 7B, the number of P-wave fragments and the duration of P-wave fragments in leads XY are almost the same as the number of P-wave fragments and the duration of P-wave fragments in leads XYZ in all of PAF, AC, and YC, and the number of P-wave fragments and the duration of P-wave fragments of a patient with atrial fibrillation are both larger than those of a healthy person.

That is, it is confirmed that the number and duration of P-wave fragments calculated in only leads XY orthogonal on the plane including the body-axis direction and the left-right direction can be used for determination of development of paroxysmal atrial fibrillation.

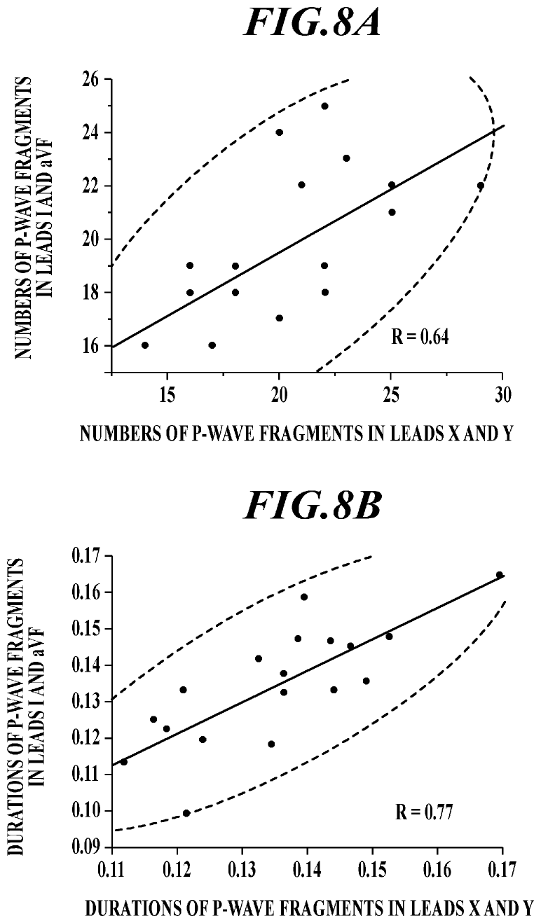

The inventors of the present invention calculated the numbers and durations of P-wave fragments of multiple healthy persons and patients with atrial fibrillation in leads XY and in lead I and lead aVF of a 12-lead ECG machine, and evaluated whether there was a correlation. The evaluation results are shown in FIGS. 8A to 10B.

FIG. 8A is a scatter plot showing correlations between the numbers of P-wave fragments in leads XY and the numbers of P-wave fragments in leads I and aVF of a 12-lead ECG machine. FIG. 8B is a scatter plot showing correlations between the durations of P-wave fragments in leads XY and the durations of P-wave fragments in leads I and aVF of a 12-lead ECG machine. As shown in FIG. 8A, the correlation coefficient of the number of P-wave fragments in leads XY and the number of P-wave fragments in leads I and aVF of a 12-lead ECG machine was 0.64, which indicated a correlation. As shown in FIG. 8B, the correlation coefficient of the duration of P-wave fragments in leads XY and the duration of P-wave fragments in leads I and aVF of a 12-lead ECG machine was 0.77, which indicated a correlation.

In addition, the inventors of the present invention evaluated whether there was a correlation between the number and duration of P-wave fragments in leads XY and the numbers and durations of P-wave fragments in leads II and aVL, and leads III and aVR so as to find out whether there is a correlation between the number and duration of P-wave fragments in leads XY and the numbers and durations of P-wave fragments in leads II and aVL, and leads III and aVR.

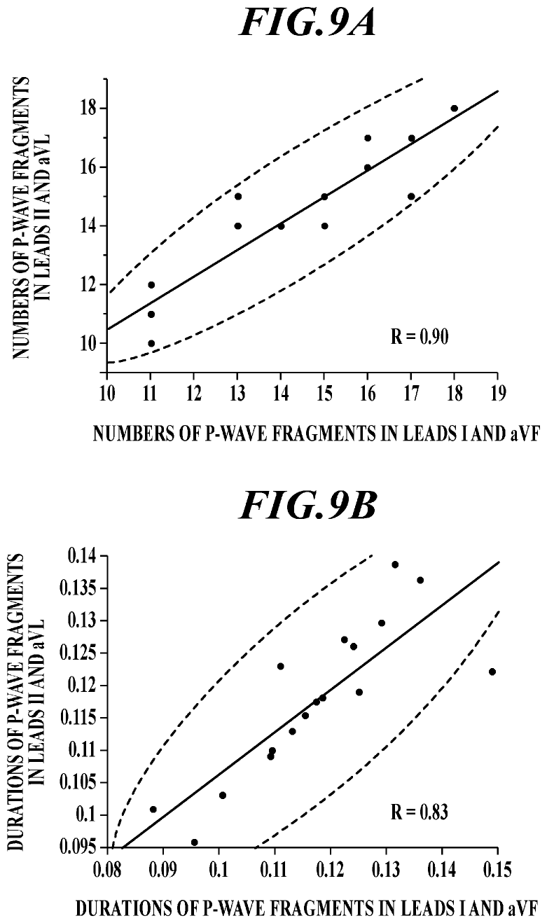

FIG. 9A is a scatter plot showing correlations between the numbers of P-wave fragments in leads I and aVF and the numbers of P-wave fragments in leads II and aVL of a 12-lead ECG machine. FIG. 9B is a scatter plot showing correlations between the durations of P-wave fragments in leads I and aVF and the durations of P-wave fragments in leads II and aVL of a 12-lead ECG machine. As shown in FIG. 9A, the correlation coefficient of the number of P-wave fragments in leads I and aVF and the number of P-wave fragments in leads II and aVL of a 12-lead ECG machine was 0.90, which indicated a correlation. As shown in FIG. 9B, the correlation coefficient of the duration of P-wave fragments in leads I and aVF and the duration of P-wave fragments in leads II and aVL of a 12-lead ECG machine was 0.83, which indicated a correlation. That is, there was a correlation between the number and duration of P-wave fragments in leads XY and the number and duration of P-wave fragments in leads II and aVL.

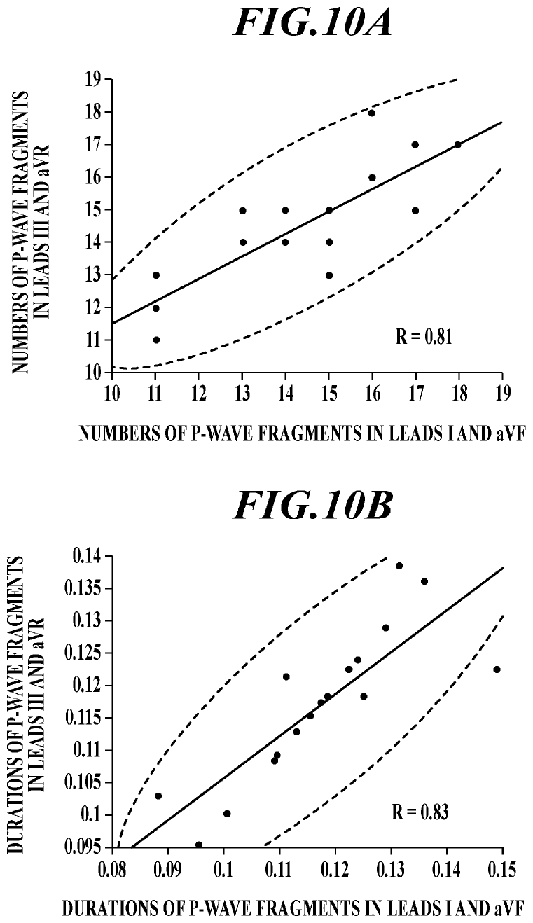

FIG. 10A is a scatter plot showing correlations between the numbers of P-wave fragments in leads I and aVF and the numbers of P-wave fragments in leads III and aVR of a 12-lead ECG machine. FIG. 10B is a scatter plot showing correlations between the durations of P-wave fragments in leads I and aVF and the durations of P-wave fragments in leads III and aVR of a 12-lead ECG machine. As shown in FIG. 10A, the correlation coefficient of the number of P-wave fragments in leads I and aVF and the number of P-wave fragments in leads III and aVR of a 12-lead ECG machine was 0.81, which indicated a correlation. As shown in FIG. 10B, the correlation coefficient of the duration of P-wave fragments in leads I and aVF and the duration of P-wave fragments in leads III and aVR of a 12-lead ECG machine was 0.83, which indicated a correlation. That is, there was a correlation between the number and duration of P-wave fragments in leads XY and the number and duration of P-wave fragments in leads III and aVR.

On the basis of the above, the number and duration of P-wave fragments calculated in leads I and aVF only, II and aVL only, and III and aVR only of a 12-lead ECG machine can be used for determination of development of paroxysmal atrial fibrillation.

An electrocardiography is a non-invasive examination that can capture the state of the entire heart on a macro scale. An examination using a 12-lead ECG is inexpensive and widely used in the medical practice, and a lot of subjects can easily undergo the examination. Thus, unlike Holter ECG that requires by 24-hour measurement, it is not necessary to perform long-time measurement. In the above-described atrial fibrillation analysis process A, the possibility of development of atrial fibrillation is analyzed from the ECG data in two leads orthogonal on the plane including the body-axis direction and the left-right direction while no symptoms are presented but not from the ECG data in a lead in the front-back direction with respect to the body. Thus, it is possible to perform analysis by the ECG data using limb leads of a 12-lead ECG that is easily measured in particular, and it is possible to accurately determine the possibility of development of atrial fibrillation with a non-invasive, inexpensive, easy, and short-time examination, even while no symptoms are presented. As a result, early diagnosis of atrial fibrillation is realized.

As the 12-lead ECG has been widely spread for a while as described above, a large amount of data in the past exist. Thus, as the ECG data of patients with atrial fibrillation and the ECG data of healthy persons in the past are used, it is possible to more accurately calculate thresholds using the positioning of P-waves and determination of P-wave fragments and thresholds for analyzing the possibility of development of atrial fibrillation and accurately estimate the possibility of development of atrial fibrillation. That is, it is possible to estimate the possibility of development of atrial fibrillation from the 12-lead ECG measurement data in the past without measuring the ECG anew. In addition, as a large amount of the 12-lead ECG measurement data in the past is input to the software or AI (machine learning) and analyzed for utilization, it is also possible to accurately estimate the possibility of development of atrial fibrillation without collecting data from a lot of examinations from now.

For example, in a case where measurement for a few minutes (for example, two minutes) at Step S1 described above is performed and analysis using two-minute ECG data is performed, short-time ECG data in the past (for example, ten seconds) may not be sufficient in regard of measurement time. In that case, multiple ECG data obtained by two-minute measurement is input to the AI, and a machine learning model for estimating two-minute ECG data from ten-second ECG data is generated. Ten-second ECG data in the past is input to the machine learning model to estimate two-minute ECG data, and thereby it is possible to utilize the short-time ECG data in the past for improvement of the accuracy of the possibility for development of atrial fibrillation. The AI may be realized by a cooperation of the controller 11 and the instruction or by a dedicated hardware.

Also, in a case where an XYZ-lead ECG machine is used, measurement in lead Z is not necessary, and an electrode dedicated to lead Z (a dedicated electrode different from those for lead X and lead Y) is not necessarily provided on the ECG measurer, which can make the device configuration inexpensive. Further, as it is not necessary to use the ECG data in lead Z, it is possible to reduce time spent on measurement, burden on a patient, analysis processing time, processing load, and the like.

### Second Embodiment

Next, the second embodiment of the present invention is described.

In the first embodiment, described is an example of analyzing the possibility of development of atrial fibrillation using the ECG data only in two leads orthogonal on the plane including the body-axis direction and the left-right direction with respect to the subject. In the second embodiment, described is an example of evaluating the possibility of development of atrial fibrillation from only the ECG data in one lead on the plane including the body-axis direction and the left-right direction with respect to the subject.

The components in the second embodiment are substantially the same as those described with reference to FIG. 1, but in this embodiment, the ECG measurer 15 can measure only ECG data in one predetermined lead on the plane including the body-axis direction and the left-right direction with respect to the subject. Thus, in this embodiment, the ECG measurer 15 measures only ECG data in one predetermined lead (for example, from the left-right angle) on the plane including the body-axis direction and the left-right direction with respect to the subject. In that case, the ECG measurer 15 may be a device of a wristband type or wristwatch type to be worn on the wrist because the burden on the subject is small.

The rest of the configuration of the second embodiment is the same as that described in the first embodiment and shares the description thereof. Hereinafter, the actions in the second embodiment are described.

Next, the actions of the atrial fibrillation analysis device 1 in the second embodiment are described.

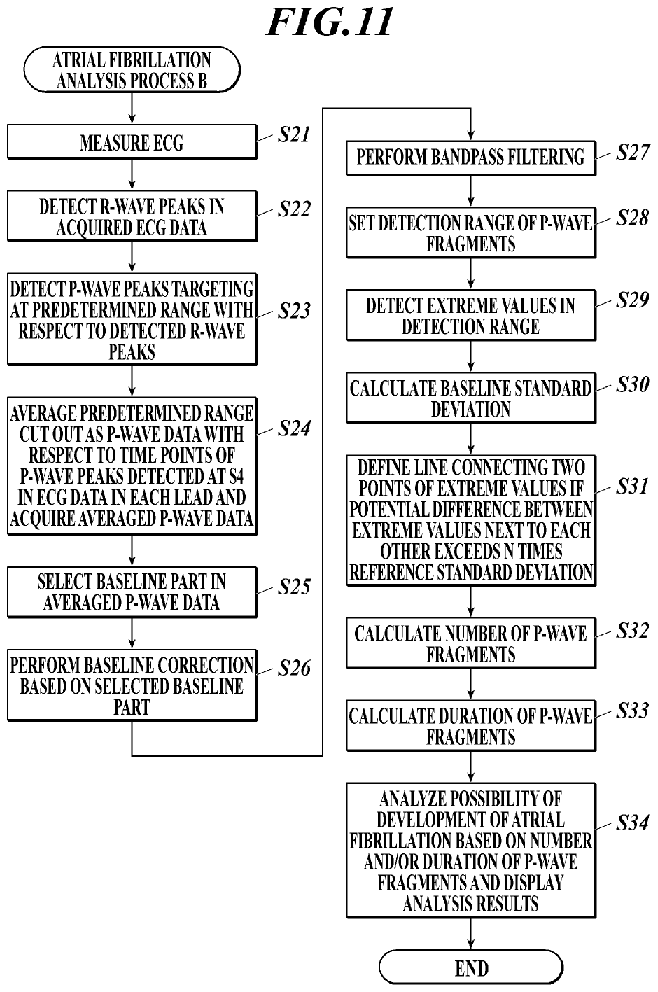

FIG. 11 is a flowchart showing a flow of the atrial fibrillation analysis process (referred to as the atrial fibrillation analysis process B) executed by the controller 11 of the atrial fibrillation analysis device 1. The atrial fibrillation analysis process B is executed by the CPU of the controller 11 in cooperation with the instruction stored in the storage 12 according to the operation of the operation interface 13.

First, the controller 11 causes the ECG measurer 15 to measure an electrocardiogram of the subject to record ECG data in a sinus rhythm (while no symptoms are presented) (Step S21).

The number of heartbeats to be measured and the measurement time are the same as those described at Step S1 in FIG. 2.

Next, the controller 11 detects R-wave peaks in the ECG data acquired by the ECG measurement (Step S22).

Next, the controller 11 detects P-wave peaks targeting at a predetermined range with respect to each detected R-wave peak (Step S23).

The predetermined range targeted for detection of P-wave peaks is defined experimentally and empirically as a range where P-wave peaks exist, which is a range of −50 to −200 mS with respect to each R-wave peak, for example.

Next, the controller 11 cuts out the predetermined range with time points of detected P-wave peaks as references in the ECG data, and averages them to calculate the averaged P-wave data (Step S24).

The predetermined range cut out as P-wave peaks is defined experimentally and empirically as a range of P waves and baselines before and after the P waves, which is a range of −300 to +150 mS with respect to each R-wave peak, for example.

Next, the controller 11 selects the baseline part in the averaged P-wave data (Step S25).

For example, the controller 11 selects the predetermined range as the baseline with respect to each P-wave peak (for example, −200 to −100 mS). The range selected as the baseline is defined experimentally and empirically as a range where the baseline exists. The baseline part may be selected by the user.

Next, the controller 11 performs the baseline correction of the averaged P-wave data based on the selected baseline part (Step S26).

For example, the averaged value of the selected baseline part is calculated and subtracted from the waveform to perform the baseline correction. As a result of this, the value of the baseline part can be substantially 0.

Next, the controller 11 performs bandpass filtering on the averaged waveform after the baseline correction (Step S27). The frequency range to pass is preferably 30 to 300 Hz and more preferably 40 to 150 Hz.

Next, the controller 11 sets the detection range of P-wave fragments in the averaged P-wave data after the bandpass filtering (Step S28).

For example, the part right after the baseline part selected at Step S25 and right before the QRS complex in the averaged P-wave data after the bandpass filtering is set as the detection range of a P-wave fragment.

Next, the controller 11 detects the extreme values in the detection range of P-wave fragments (Step S29).

Next, the controller 11 calculates the standard deviation (baseline standard deviation) of the values at the baseline part in the averaged P-wave data after the bandpass filtering (Step S30).

Next, if the potential difference between the extreme values next to each other detected at Step S30 exceeds n times the baseline standard deviation (n is a positive number), the controller 11 defines the line connecting the two points as a P-wave fragment (Step S31).

n is a value calculated based on the experiment, and is 3, for example.

Next, the controller 11 calculates the number of P-wave fragments (Step S32).

Next, the controller 11 calculates the time from the start point to the end point of P-wave fragments (duration of P-wave fragments) (Step S33).

The controller 11 analyzes the possibility of development of atrial fibrillation based on the number and/or duration of P-wave fragments, displays the analysis results on the display 14 (Step S34), and ends the atrial fibrillation analysis process B.

Step S34 is the same as Step S16 in FIG. 2, for example.

In the above-described second embodiment, the possibility of development of atrial fibrillation is analyzed using the ECG data only in one lead (for example, from the left-right angle) on the plane including the body-axis direction and the left-right direction with respect to the subject. Thus, it is possible to use measurement results by a simple ECG examination using a device of a wristband type or wristwatch type because the burden on the subject is small. On the other hand, the analysis accuracy is spared in comparison to the cases using the electrocardiograms in two leads as in the first embodiment.

For example, ECG measurement in a lead for analysis in the above-described atrial fibrillation analysis process B (a lead used in analysis) and a lead orthogonal to that lead for analysis on the plane including the body-axis direction and the left-right direction with respect to the subject may be performed multiple times, and the numbers and/or durations of P-wave fragments calculated using the ECG data in the lead for analysis and the numbers and/or durations of P-wave fragments calculated using the ECG data in two leads of the lead for analysis and the lead orthogonal to that lead may be input to the AI constructed in the atrial fibrillation analysis device 1 and learned to generate a machine learning model. The controller 11 may input the numbers and/or durations of P-wave fragments calculated in the process at Steps S32 to S33 in FIG. 11 to the above-described machine learning model, estimate the number and/or duration of P-wave fragments calculated using the ECG data in two leads, and analyze the possibility of development of atrial fibrillation based on the estimated number and/or duration of P-wave fragments. This makes it possible to acquire the effects similar to the first embodiment more easily.

As described hereinbefore, in the atrial fibrillation analysis device 1, the controller 11 acquires the P-wave data from electrocardiograms in one lead on the plane including the body-axis direction and the left-right direction with respect to the subject only or electrocardiograms in two leads orthogonal on that plane only, and extracts P-wave fragments from the acquired P-wave data. The possibility of development of atrial fibrillation is thereby analyzed based on the number and/or duration of P-wave fragments.

Therefore, the possibility of development of atrial fibrillation is analyzed by the ECG data in one lead or two leads orthogonal on the plane including the body-axis direction and the left-right direction while no symptoms are presented, but not by the ECG data in a lead in the front-back direction with respect to the body. Thus, it is possible to perform analysis by the ECG data using limb leads of a 12-lead ECG that is easily measured in particular, and it is possible to accurately determine the possibility of development of atrial fibrillation with a non-invasive, inexpensive, easy, and short-time examination, even while no symptoms are presented. As a result, early diagnosis of atrial fibrillation is possible.

The above-described embodiments are mere examples of implementation of the present invention, and do not limit the present invention.

For example, in the above-described first and second embodiments, the atrial fibrillation analysis device 1 analyzes the possibility of development of atrial fibrillation from ECG data in one lead on the plane including the body-axis direction and the left-right direction with respect to the subject only or ECG data in two leads orthogonal on the plane including the body-axis direction and the left-right direction with respect to the subject only, and the device and the atrial fibrillation analysis method on the device are described. However, the device and analysis method (P-wave fragment analysis device and P-wave fragment analysis method) may calculate the number and/or duration of P-wave fragments from ECG data in one lead on the plane including the body-axis direction and the left-right direction with respect to the subject only or ECG data in two leads orthogonal on the plane including the body-axis direction and the left-right direction with respect to the subject only.

In the above-described second embodiment, the ECG measurer 15 measures ECG data in one predetermined lead on the plane including the body-axis direction and the left-right direction with respect to the subject (for example, from the left-right angle) only, but the present invention is not limited to this example. An ECG machine that can acquire electrocardiograms in multiple leads may be used. For example, a 12-lead ECG machine or an XYZ-lead ECG machine may be used. Then, the controller 11 may acquire the ECG data in a predetermined lead from the measured ECG data and analyze the P-wave fragments and the possibility of development of atrial fibrillation.

The above description discloses an example of using a hard disk, a semiconductor nonvolatile memory and the like as the computer readable medium of the instruction according to one or more embodiments. However the present invention is not limited to the example. A portable recording medium such as a CD-ROM is applicable as other computer readable mediums. A carrier wave is also applied as a medium providing the instruction data according to one or more embodiments via a communication line.

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

### INDUSTRIAL APPLICABILITY

One or more embodiments are applicable to the medical field.

### REFERENCE SIGNS LIST

1 Atrial Fibrillation Analysis Device
11 Controller
12 Storage
13 Operation Interface
14 Display
15 ECG Measurer
16 Communication Unit
17 Bus

1. An atrial fibrillation analysis device comprising:
a hardware processor that:
acquires P-wave data from only one of either:
a first electrocardiogram in a lead of one direction on a plane including a body-axis direction and a left-right direction with respect to a subject, or
a second electrocardiogram in leads of two directions orthogonal to each other on the plane;
extracts P-wave fragments from the acquired P-wave data; and
analyzes a possibility of development of atrial fibrillation based on at least one of a number of the P-wave fragments and a duration of the P-wave fragments.

2. The atrial fibrillation analysis device according to claim 1, further comprising:
an ECG measurer that measures the first or second electrocardiogram.

3. The atrial fibrillation analysis device according to claim 1, wherein
the hardware processor further:
acquires a plurality of pieces of P-wave data from the first or second electrocardiogram,
averages the plurality of pieces of P-wave data to calculate averaged P-wave data,
extracts an extreme value from the averaged P-wave data, and
in response to a potential difference between the extreme value and an adjacent extreme value exceeding a predetermined value, extracts a line connecting the extreme value and the adjacent extreme value as a P-wave fragment.

4. The atrial fibrillation analysis device according to claim 3, wherein
in response to acquisition of the P-wave data from the second electrocardiogram, the hardware processor calculates the averaged P-wave data by averaging the plurality of pieces of P-wave data for each of the leads to calculate a root mean square.

5. The atrial fibrillation analysis device according to claim 3, wherein
the hardware processor filters out a predetermined range of frequency from the averaged P-wave data and extracts the P-wave fragments from the filtered averaged P-wave data.

6. An atrial fibrillation analysis method comprising:
acquiring P-wave data from only one of either:
an electrocardiogram in a lead of one direction on a plane including a body-axis direction and a left-right direction with respect to a subject, or
an electrocardiogram in leads of two directions orthogonal to each other on the plane;
extracting P-wave fragments from the acquired P-wave data; and
analyzing a possibility of development of atrial fibrillation based on at least one of a number of the P-wave fragments and a duration of the P-wave fragments.

7. A non-transitory computer-readable storage medium storing a program that causes a computer to:
acquire P-wave data from only one of either:
an electrocardiogram in a lead of one direction on a plane including a body-axis direction and a left-right direction with respect to a subject, or
an electrocardiogram in leads of two directions orthogonal to each other on the plane;
extract P-wave fragments from the acquired P-wave data; and
analyze a possibility of development of atrial fibrillation based on at least one of a number of the P-wave fragments and a duration of the P-wave fragments.
